(19) United States
(12) Patent Application Publication
(10) Pub. No.: US 2021/0162232 A1
(43) Pub. Date: Jun. 3, 2021

(54) APPARATUS FOR BIOPHOTONIC TISSUE TREATMENTS

(71) Applicant: Hyperion Med S.R.L., Cascina (PI) (IT)

(72) Inventors: Daniele Bani, Firenze (FI) (IT); Marco Giannelli, Firenze (FI) (IT)

(73) Assignee: Hyperion Med S.R.L., Cascina (PI) (IT)

(21) Appl. No.: 17/047,756

(22) PCT Filed: May 17, 2019
(86) PCT No.: PCT/IT2019/050107

§ 371 Date: Oct. 15, 2020

(30) Foreign Application Priority Data
May 24, 2018 (IT) 102018000005693

(51) Int. Cl. A61N 5/06

(52) U.S. Cl. CPC A61N 5/0624; A61N 5/0603; A61N 5/0616; A61N 2005/0645; A61B 5/01; A61N 2005/0627; A61N 2005/0606; A61N 2005/0659; A61N 2005/0663; A61N 2005/063

(57) ABSTRACT

An apparatus for biophotonic tissue treatments comprising a wearable light emitter device, suitable to be configured to be applied to a body portion to be submitted to treatment. The light emitter device comprising a closed chamber, light guides being formed at the inside in order to transfer a preset-frequency light radiation at pre-fixed treatment points of the device. The light guides are connected to at least an optical fibre that direct thereof the light generated by at least a light radiation source arranged in a control unit for controlling the emission of light radiation. The control unit is operated by an operation unit for managing the treatment and for monitoring its implementation thereof according to set up parameters. The light guides being formed during the same production process and with the same material as the light emitter device.

### FIELD OF THE INVENTION

This invention generally relates to the sector of cellular photodynamic treatments and more specifically to biophotonic tissue treatments, in particular tissues of a human body. More specifically, the invention relates to an apparatus for biophotonic tissue treatments, in particular one provided with applicator means for applying electromagnetic energy to a given tissue, said applicators being of a customizable and wearable type.

### PRESENT STATUS OF THE ART

Therapeutic treatments based on the use of electromagnetic energy, in particular in the form of light energy at different wavelengths, are known and widely used for decades in different fields of medicine. The therapeutic use of light is very wide in medicine and the most representative articles concerning applications in different pathologies in the recent years can be found in the following example, non-limitative list:
1) Cieplik F, Buchalla W, Hellwig E, Al-Ahmad A, Hiller K A, Maisch T, Karygianni L. Antimicrobial photodynamic therapy as an adjunct for treatment of deep carious lesions-A systematic review. Photodiagnosis Photodyn Ther. 2017 June;18:54-62.
2) Tavares L J, Pavarina A C, Vergani C E, de Avila E D. The impact of antimicrobial photodynamic therapy on peri-implant disease: What mechanisms are involved in this novel treatment? Photodiagnosis Photodyn Ther. 2017 March; 17:236-244.
3) Hamblin M R. Antimicrobial photodynamic inactivation: a bright new technique to kill resistant microbes. Curr Opin Microbiol. 2016 October;33:67-73.
4) Fracalossi C, Nagata J Y, Pellosi D S, Terada R S, Hioka N, Baesso M L, Sato F, Rosalen P L, Caetano W, Fujimaki M. Singlet oxygen production by combining erythrosine and halogen light for photodynamic inactivation of Streptococcus mutans. Photodiagnosis Photodyn Ther. 2016 September; 15:127-32.
5) Arany P R. Craniofacial Wound Healing with Photobiomodulation Therapy: New Insights and Current Challenges. J Dent Res. 2016 August;95(9):977-84.
6) Kuffler D P. Photobiomodulation in promoting wound healing: a review. Regen Med. 2016 January;11(1):107-22.
7) Yadav A, Gupta A. Noninvasive red and near-infrared wavelength-induced photobiomodulation: promoting impaired cutaneous wound healing. Photodermatol Photoimmunol Photomed. 2017 January;33(1):4-13.
8) Hamblin M R. Shining light on the head: Photobiomodulation for brain disorders. BBA Clin. 2016 Oct. 1;6:113-124
9) Chow R T, Armati P J. Photobiomodulation: Implications for Anesthesia and Pain Relief. Photomed Laser Surg. 2016 December;34(12):599-609.
10) Chiari S. Photobiomodulation and Lasers. Front Oral Biol. 2016;18:118-23.
11) Pandeshwar P, Roa M D, Das R, Shastry S P, Kaul R, Srinivasreddy M B. Photobiomodulation in oral medicine: a review. J Investig Clin Dent. 2016 May;7(2):114-26
12) Akram Z, Al-Shareef S A, Daood U, Asiri F Y, Shah A H, AlQahtani M A, Vohra F, Javed F. Bactericidal efficacy of photodynamic therapy against periodontal pathogens in periodontal disease: a systematic review. Photomed Laser Surg. 2016 April;34(4):137-49
13) Qiushuo Sun, Yuezhi He, Kai Liu, Shuting Fan, Edward P. J. Parrott, Emma Pickwell-MacPherson, “Recent advances in terahertz technology for biomedicai applications,” Quant Imaging Med Surg. 2017 June;7(3):345-355. doi: 10.21037/qims.2017.06.02.

See also, as an example, U.S. Pat. No. 7,033,381.

However, there are some sectors wherein such diffusion is very limited, but an application of this type of treatments might lead to numerous advantages, especially in so little conspicuous and/or everyday life ordinary areas as to gain little evidence, but wherein pathologies have considerable social impact and sanitary costs. Let's mention among these, for example, parodontal and peri-implant diseases, diabetic ulcers, skin infections and sores, etc. In these sectors, applying targeted photonic techniques might lead to significant progresses in therapies, improved life conditions for patients, reduced costs and social impact. In particular, the antiseptic effect demonstrated by phototherapy and photodynamic therapy on different pathogenic species complies with a requirement of limiting the use of conventional antibiotics and meeting the WHO (World Health Organization)'s recommendations, to prevent the antibiotic-resistance phenomenon, which is considered a priority in the EU's guidelines with regard to public health.

In general, the light energy produced by lasers, laser diodes, or light emitting diodes (LEDs) is used for this type of treatments. Laser- and LED-based treatments are usually made in surgeries and/or at the premises of specialized structures. This is unavoidable for surgical applications and, likewise, a medical control of the therapy is necessary during the administration period. This makes maintenance sessions little frequent over time. Apparatuses and devices of this type for outpatient treatments are described, for instance, in document U.S. Pat. No. 4,852,549. On the other hand, applications such as photodynamic, bio-inductive, anti-inflammatory, and/or decontamination therapies might get better and/or more lasting results if repeated at a greater frequency.

An availability of apparatuses and devices for a personal and domiciliary care might represent a therapeutic improvement, in that it would permit a more frequent application of therapies. In the dental sector, devices have been proposed suitable for being applied to a patient's dental arch, wherein the light sources are inserted. For example, document WO98/06456 discloses an apparatus for treating gengival and periodontal diseases, which comprises a U-shape applicator defining a groove. Light is emitted from points spaced from each other and arranged adjacent to the inner walls of the applicator which delimit the groove in such a way that light is incident onto a gum. An external light source is used and recourse is made to an optical fibre beam to direct light from the outside of the oral cavity to points spaced along the inner walls of the applicator's groove, at which the ends of corresponding optical fibres are located. Alternatively, LEDs spaced from each other and receiving power from an external electric source can be arranged close to the inner walls of the groove to emit light towards a gum. The positioning of the optical fibres in the applicator is a laborious operation, whereas the use of LEDs results in an unacceptable overheating of the oral region concerned.

Document WO2013155632, on behalf of Biolux Research Ltd. (Canada), discloses an orthodontic therapy apparatus for domiciliary use equipped with a light emitter emitting light towards a region associated with the alveolar soft tissue and an electronic circuit for controlling the emitter. Light administration is made in such a way as to possibly modify the speed of movement of teeth and increase rate of healing. The light emitter is arranged in a housing, which can be tailored to a given patient.

Another example of use of electromagnetic radiation is in tooth whitening apparatuses, in association with a whitening substance which can be activated by said radiation. Tooth whitening apparatuses of this type are illustrated, for instance, in documents WO2006020128, WO20060148972 and US2013045457A1. In these apparatuses, a plurality of LEDs, suitable for emitting light in the blue spectrum at a wavelength ranging, for instance, from 430 to 450 nm, is arranged on a shaped support to fit a user's dental arch. A substance generally containing a peroxide, for instance in the form of a gel, is arranged around the teeth so that, whenever the blue light produced by the LEDs is directed to the teeth, it activates the peroxide which results in whitening the teeth in contact therewith. The LED support is possibly in the form of a “bite”, in order for it to be easily held in the mouth with the teeth. The problem of overheating due to the LEDs is also present in this case.

Document WO 2017044931 discloses an intra-oral phototherapy device configured for receiving light from an associated light source and propagating it in a patient's oral cavity by way of a total inner reflection. This device comprises light guides, connected to the light source via optical fibre, extending from a support intended for being positioned in a patient's mouth and oriented towards regions of the oral cavity to be treated. The light guides are enclosed within transparent coating sleeves to prevent the light guides from getting in contact with saliva or inner parts of the mouth. In this solution, the need for using sleeves and/or sheaths in order to provide for an appropriate light transmission constitutes a constructional complication and possible difficulties in the use of the device.

The apparatuses for biophotonic tissue treatments according to the known art still present many problematic aspects of both structural and functional natures which prevent a widespread, domestic, and customized use thereof or limit such use to very specific applications.

So, a need is very much felt for an apparatus for biophotonic tissue treatments that is customizable and features a high versatility of use and can be produced at a reduced cost. An apparatus of this type would open the way to the continuation of the various treatments at domiciliary level, directly performed by a patient, thus allowing for more frequent treatments hence a faster healing and guaranteeing a conservation of the obtained results over time.

### SUMMARY OF THE INVENTION

The general object of the present invention is to provide an apparatus for biophotonic tissue treatments that allows to overcome the above-mentioned drawbacks of the known art.

A particular object of the present invention is to provide an apparatus of the mentioned type that is wearable and consequently can make it possible to perform said treatments at a domestic level.

Another object of the present invention is to provide an apparatus of the mentioned type that can be customized for the specific requirements of a subject to be treated.

A further object of the present invention is to provide an apparatus of the mentioned type wherein transmission of light radiation takes place under internal total refection conditions.

Another object of the present invention is to provide an apparatus of the mentioned type that allows biophotonic tissue treatments at different wavelengths of the light radiation.

A further object of the present invention is to provide an apparatus of the mentioned type that can be manufactured at reduced costs.

These objects are achieved by an apparatus for biophotonic tissue treatments according to the present invention, whose basic characteristics are set forth in claim 1. Further important characteristics are set forth in the dependent claims.

According to an important characteristic of the invention, the apparatus for biophotonic tissue treatments comprises a wearable light emitter device, suitable for being configured in such a way as to be possibly applied to a body portion to be treated. The light emitter device comprises a closed chamber, internally to which light guides are derived whose distal ends surface from at prefixed treatment points, and proximal ends merging to an inlet connector where a connection takes place between the proximal ends of said light guides and at least one optical fibre. The optical fibre directs the light generated by at least one light radiation source arranged in a light radiation emission control unit, the control unit being operated by an operation unit which manages said treatment and supervises the implementation thereof according to set up parameters, the light guides being formed during the same production process and being made from the same material as the light emitter device.

According to a particularly preferred embodiment of the invention, the light emitter device comprises two shells delimiting said closed chamber, said light guides extending within said chamber along one of said shells and ending with their distal ends at said prefixed treatment points defined on the other one of said shells. Thanks to this configuration of the light emitter device, the shells constitute a mechanical support and a functional protection for the light guides, by making air fill the surrounding space so that the difference of the index of refraction with the light guides provides for a correct propagation of the radiation, without any dispersions, up to the proximal ends at the points to be treated.

### BRIEF DESCRIPTION OF THE DRAWINGS

These characteristics and others, as well as the advantages of the apparatus for biophotonic tissue treatments according to the present invention will be apparent from the following description of its embodiments, which are given for explanatory non-limitative purposes only, with reference to the attached drawings, wherein:

FIG. 1 is a schematic perspective view of an applicator of the apparatus for biophotonic tissue treatments according to one embodiment of the invention;

FIG. 2 is a schematic cross-sectional view of the applicator of FIG. 1 according to the arrows II-II in FIG. 1;

FIG. 3 is schematic partially cross-sectional view according to the arrows III-III in FIG. 2;

FIG. 4 is a schematic block diagram of the apparatus according to the invention;

FIG. 5 is a perspective view of a 3D model of the applicator of the apparatus according to the invention;

FIG. 6 is a schematic exploded cross-sectional view of an applicator of the apparatus for biophotonic tissue treatments according to another embodiment of the invention;

FIGS. 7a and 7b are schematic perspective views of a first and second shells of the applicator of FIG. 6, shown in an overturned position as compared to the view in FIG. 6;

FIG. 8 is a schematic perspective view of the applicator of FIG. 6 in an overturned position as compared to the view in FIG. 6;

FIG. 9 is a schematic perspective view of the second shell, the inside being modeled by the prints of the teeth of a dental arch on which it shall be applied;

FIG. 10 shows a block diagram of the control unit of the apparatus according to the invention;

FIG. 11 shows a block diagram of an operation unit of the apparatus according to the invention;

FIGS. 12 and 13 show two different perspective views of the light emitter device of the apparatus according to the invention suitable for treating onychomycosis;

FIGS. 14 and 15 show two different views of the light emitter device of the apparatus according to the invention suitable for treating foot ulcers and sores;

FIG. 16 show a perspective view of the light emitter device of the apparatus according to the invention suitable for endovaginal treatments.

### DETAILED DESCRIPTION OF THE INVENTION

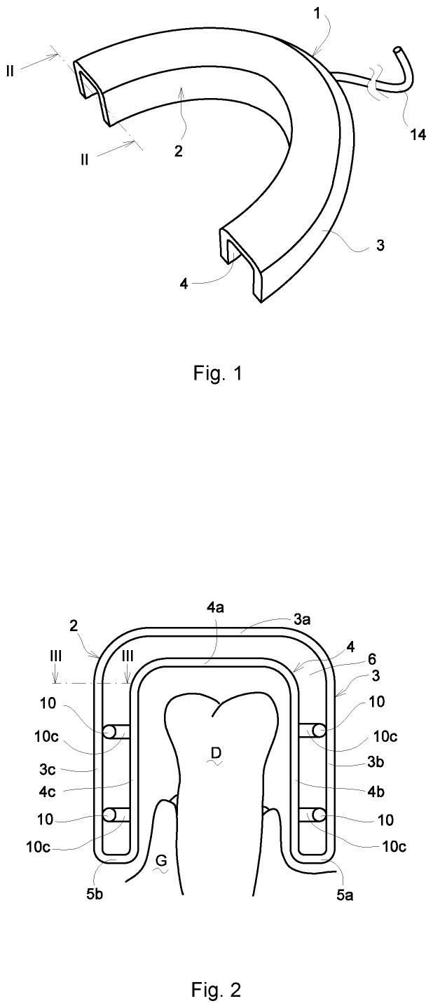

With reference to FIGS. 1 and 2, the reference numeral 1 generally indicates a light emitter device for applying light energy to a human body's tissue portion which needs a treatment, simply referred to as applicator in the following description for the sake of simplicity. In the embodiment here shown, the applicator schematically takes the shape of a “bite” formed, as a whole, of an arcuate plate having a substantially U-shaped cross-section, configured in such a way as to be possibly applied to a dental arch to cover the teeth and, if necessary, to also extend above the gingival system. Bites are widely known and used as therapeutic aids for solving or attenuating disorders related to an incorrect dental layout, for whitening teeth, and for other functions. In the figures, the applicator is shown in a fully schematic form and its respective component parts are not in scale to each other for the sake of clarity, but it shall be understood that it will be modeled and shaped in its practical implementation in a known way for fitting the shape of the dental arch and of the individual teeth on which it shall be applied in use.

In the embodiment of the invention illustrated in FIGS. 1 and 2, the applicator 1 is formed of a shaped plate 2, typically featuring an arcuate shape and having a substantially U-shaped cross-section. The shaped plate 2 is formed of a first shell 3 and a second shell 4, both featuring an arcuate shape and substantially U-shaped. With special reference to FIG. 2, which also schematically shows a tooth D and a gum G which the tooth extends from, the first shell 3 is formed of a base 3a which, in the use of the applicator according to the invention, is intended for positioning above the dental crown, and two side walls 3b and 3c extending from opposite sides of the base 3a along the outer part and the inner part of the teeth (wherein the terms “outer” and “inner” are referred to the outside and the inside of the oral cavity respectively).

Likewise, the second shell 4 is formed of a base 4a which, in the use of the applicator according to the invention, is intended for also positioning above the dental crown, and two side walls 4b and 4c extending from opposite sides of the base 4a along the outer part and the inner part of the teeth. The second shell 4 is arranged within the first shell 3 and the respective bases 3a and 4a of the two shells, and likewise their respective side walls 3b, 4b and 3c, 4c of the two shells extend according to a substantially two by two parallel relationship. The side walls 3b and 4b and the side walls 3c and 4c of the two shells 3 and 4 link up in 5a and 5b respectively at the respective free ends of said side walls so as to join the first shell 3 to the second shell 4, which is located inside the first shell 3 and consequently is closer to the dental arch in use. The two shells 3 and 4 thus coupled together delimit an inner chamber 6 of the U-shaped plate 2 closed and isolated from the external world.

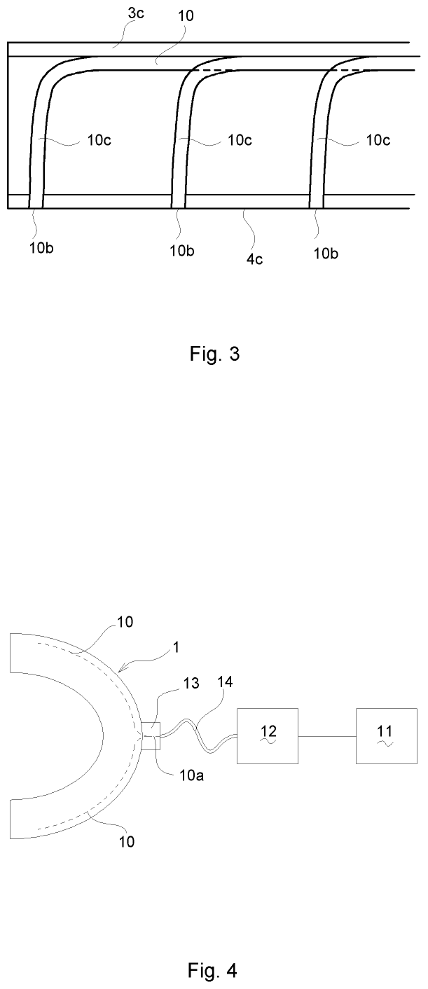

As also shown in FIG. 3, light guides 10 are provided inside the chamber 6 and along the side walls 3b and 3c of the first shell, which are schematically shown in FIGS. 2 and 3 and whose function is to direct electromagnetic energy, in the form of light, from one or several light sources external to the applicator 1 to points of use predefined on the side walls 4b and 4c of the second shell 4. In the present embodiment according to the invention, the light guides 10 feature a substantial cylindrical shape and are joined to their respective side walls 3b and 3c along a generatrix thereof. Light guides arms 10c extend from the light guides 10 which, through their distal ends 10b, carry light from the light guide 10 extending along the walls 3b, 3c of the first shell 3 to the prefixed points of the side walls 4b and 4c of the second shell 4.

Since the first shell 3 and the second shell 4, joined to each other, delimit a closed air chamber formed of the chamber 6, the light guides 10 can transmit light under internal total reflection conditions because, in this way, even though the applicator is inserted into a user's mouth, they do not get in contact with saliva or any parts of the mouth, but only with the air internal to the chamber 6, whereby light remains confined inside the guide up to its point of exit at the distal end of the light guide.

The light guides 10 are derived on the side walls 3b and 3c of the first shell 3 and are built in one molding process together with the first shell 3 and the second shell 4 to form one piece integral therewith. The preferred molding processes include 3D molding such as, for example but non-limiting purposes only, FDM, Polijet, SLA stereolithography, SLS, etc. molding, and a classic molding based on the use of molds. The materials usable are selected among those normally used for these molding techniques, provided they are biocompatible and optically transparent in order for them to be suitable for light transmission. The materials usable can also be relatively soft and flexible. A polymeric material meeting these requirements is the polycarbonate. Other polymeric materials usable are, for example but non-limiting purposes only, polystyrene, polymethylmethacrylate, silicone, polyolefins, nylon, ABS, polyurethane, or other biocompatible thermoplastic elastomeric materials or biocompatible photo-polymerizable resins.

As shown in FIG. 4, the light guides 10 merge with their proximal ends 10a, on a connector 13 placed in an appropriate position on the outside of the shell 3 where they are connected in a known way to the end of one or several optical fibres 14 coming from a control unit 12 also incorporating the light source(s). The control unit also comprises, for example but non-limitative purposes only, a power supply and battery charger section; a rechargeable battery; a microcontroller; a light source (laser diode and/or LED) drive section; a diagnostic signal detection and amplification section; control and security circuits; a connection to a user interface.

The light source is in the visible and near infrared portions of the spectrum, in particular in the 400-1100 nm, preferably in the 400-450 nm, 530-660 nm and 750-1100 nm portions, and most preferably in the 400-415 nm, 605-660 nm and 800-820 nm ranges.

In particular, the infrared radiation light source is such that the flux obtained is in the 10-200 J/cm² range, preferably in the 50-150 J/cm² range, and most preferably in the 70-120 J/cm² range; the red radiation light source is such that the flux obtained is in the 2-200 J/cm², preferably in the 5-100 J/cm² range, and most preferably in the 7-50 J/cm² range; the violet radiation light source is such that the flux obtained is in the 10-200 J/cm² range; preferably in the 30-100 J/cm² range; and most preferably in 50-75 J/cm² range.

LED switching on/off is controlled by the microcontroller according to the parameters set up for each specific treatment type.

The control and security circuits make it possible to verify whether the conditions under which the system operates are within the specified limits and, if necessary, suspend radiation and report the problem (e.g. fibre disconnected, temperature too high, etc.) to the user.

The control unit 12 is connected to an operation unit 11 provided with a user interface by way of which the operation of the apparatus can be managed. The user interface is formed, for example, of a touch sensitive display, push-buttons and pilot/indicators, or a mobile application via a Bluetooth connection for the most popular operating systems. The user can use this interface to interact with an operation unit 11, to enter commands, to set parameters, and to monitor the operation of the system. The operations a user can control/monitor will depend on the status of the system and on the sets-up made by a doctor.

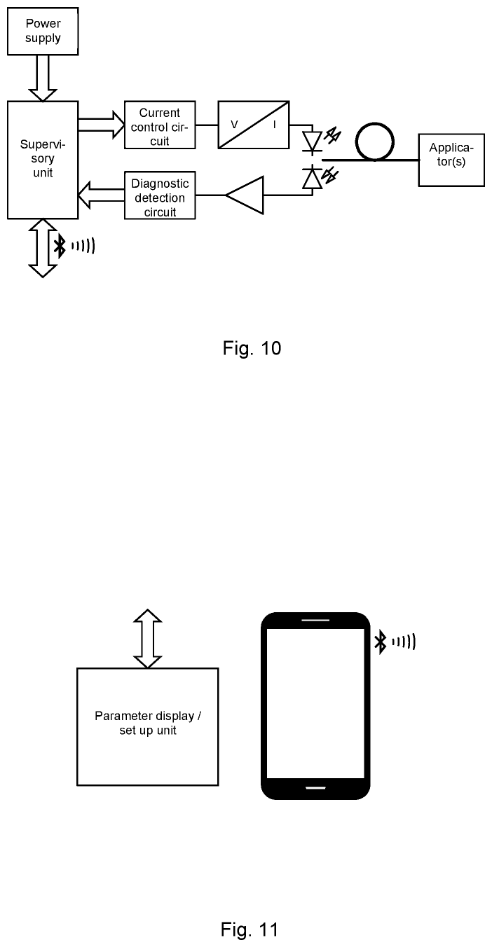

A possible implementation of the control unit 12 is shown in FIG. 10, whereas a possible implementation of the operation unit 11 is shown in FIG. 11. The dimensions of the control unit are reduced so as to make it possible for a user to “wear” it comfortably, for example by putting it in a pocket of his/her suit/dress.

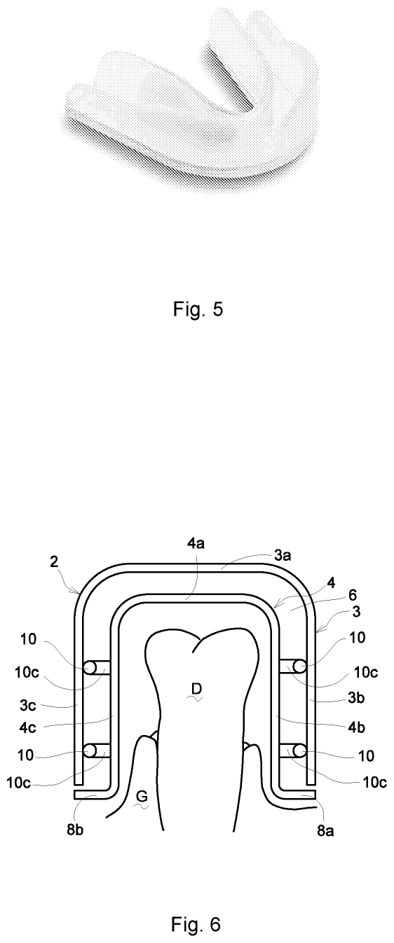

In one embodiment of the invention, the applicator 1 can be intended for a complete dental arch, as in the form schematically illustrated in FIG. 1 and as also schematically illustrated in the model shown in FIG. 5. In another embodiment. the applicator 1 can be intended for covering a portion only of a dental arch depending on the therapeutic, preventive, or cosmetic function the apparatus is intended for performing.

In another embodiment, schematically shown in FIG. 6, the substantially U-shaped plate 2 is made of two pieces intended for being joined to each other, for example by way of a hot welding, to delimit the chamber 6 internally to the shaped plate 2. More specifically, the two pieces are formed of the first shell 3 and the second shell 4 and the latter features two link-up flaps 8a and 8b at the free ends of its walls 4b and 4c, protruding outside from the second shell 4, on which the free end of the walls 3b and 3c of the first shell 3 is secured, so as to delimit, between the two shells, the closed chamber 6 within which the light guides 10 are arranged. The main advantage of such a type of solution consists in the possibility of performing treatment operations on the inner surfaces and/or the light guides 10 after their molding and subsequently coupling the two shells together.

The first and second shells 3 and 4 are manufactured by way of separated molding operations and are subsequently joined to each other by way of a welding operation or another technique, depending on the material used. In FIG. 6 the first and second shells 3 and 4 are shown in an exploded view separated from each other for illustrative reasons, but, in the practical use of the applicator according to the invention, they are actually joined together and form one piece, i.e. a shaped plate 2.

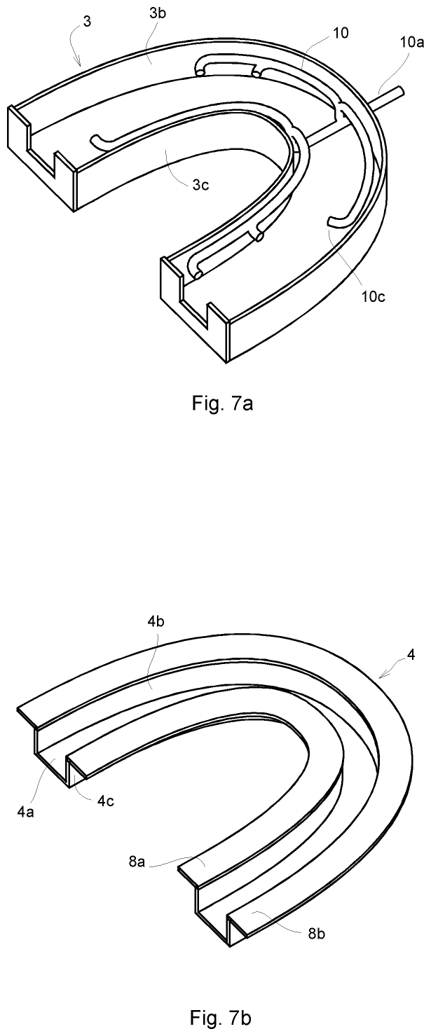

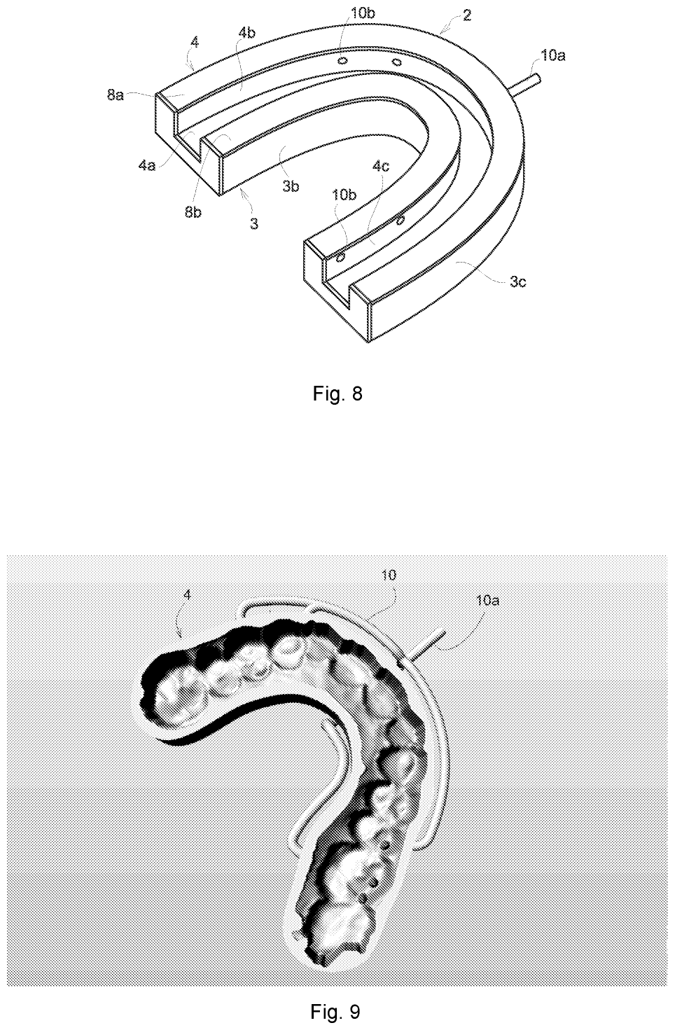

FIGS. 7a and 7b show a schematic perspective view of the first shell 3 and of the second shell 4 separated from each other and in overturned position as compared to that illustrated in FIG. 6. Conversely, FIG. 8 shows a schematic perspective view, also in an overturned position as compared to that illustrated in FIG. 6, wherein the two shells 3 and 4 have been assembled and joined together to form the applicator of the apparatus according to the invention. The light guides 10 and their respective light guide arms 10c, arranged according to a possible distribution mode, are also visible in the first shell 3. The second shell 4 is shown in FIG. 7b along with its link-up flaps 8a and 8b and its shape and dimensions are such as to be possibly engaged in the first shell 3 between the walls 3b and 3c thereof so that the free ends of the light guide arms 10c are located at the radiating points predetermined on the walls 4b and 4c of the second shell 4. When the second shell 4 is arranged in the first shell 3, the perimetric edges of the walls 3b and 3c get closer to the link-up flaps 8a, 8b in order for them to be joined therewith, for instance by way of a hot welding operation or another appropriate technique, depending on the material used.

FIG. 9 shows a model of the second shell 4 wherein the prints of the teeth of a dental arch are visible, as well as the light guides merging on the walls 4b and 4c thereof and extending along the walls 3b and 3c of the first shell 3, which is not shown in the figure in order to make the representation clearer.

An advantage offered by the apparatus according to the present invention consists of its high versatility of use. As a matter of fact, the applicator 1 can be customized both in terms of shape, which shall perfectly fit the shape of a user's teeth, and in terms of points of application of the light beams, which points are previously identified by that who performs the necessary treatment. This results from the fact that the light guides 10 are implemented in the course of the same process whereby the first shell 3 that support them is molded. For this purpose, in the moment when a digital print of the dental arch, or a portion thereof, involved in the treatment is taken, the radiating points are also marked thereon as necessary to define the corresponding light guide and its respective path. Then, by way of a CAD image, or equivalent, of the thus integrated print, the applicator is molded by using 3D techniques such as, as a non-limiting example, an FDM, Polijet, SLA stereolithography, SLS, etc. technique or a classic molding making use of a mold. The applicator thus obtained is connected to the optical fibres output from the control unit 12 via the connector 13 after checking for its wearability and, if necessary, adjusting it.

Another important advantage of the apparatus according to the present invention is in that it can be operated in a multi-spectral mode, i.e. it can support an irradiation with light radiations at three different wavelengths, namely 808 nm, 635 nm and 405 nm. These different wavelengths correspond, as known, to light radiation treatments for different indications. The 808 nm light radiation is indicated for analgesic, anti-inflammatory, and decontaminant treatments. The 635 nm light radiation is indicated for photodynamic treatments for antibacterial and decontaminating purposes on some bacterial species and fungines by applying light-activated agents having antiseptic effects, such as methylene blue, toluidine blue and other similar substances. The 635 nm radiation might also be used for biostimulation treatments for fostering healing after interventions, improving tissue regeneration, fostering bone integration in dental implants, speeding up healing of oral aphtae, etc. The 405 nm light radiation can be used for exerting an antiseptic action on some bacterial species and fungines also in combination with light-activated substances such as curcumin yellow and other similar molecules, and for helping healing of skin lesions.

In one embodiment of the apparatus according to the invention, light radiations can be applied individually; in another embodiment, they can be applied in variously combined manners.

In a further embodiment, emission can be continuous or pulsed, at a pulsing frequency in the range from 50 Hz to 50 kHz and in particular from 10 Hz to 30 kHz.

In one embodiment of the invention, optical and/or thermal sensors can be provided internally to the applicator 1 for monitoring and controlling the treatment, and also some light guides might be provided to output a light signal to be analyzed and to get diagnostic or other types of information. The light signals collected by the light guides are detected by appropriate photosensitive elements, such as for instance photodiodes, and are properly amplified and acquired by the microcontroller equipped in the control unit 12. Their subsequent processing makes it possible to get diagnostic information of interest in a tissue, such as for instance temperature, inflammatory status, color, treatment progress. Signals useful for diagnostics can feature wavelengths in the mid-wavelength infrared range, preferably in the 10-2 μm portion, in the near infrared range, preferably in the 750-980 nm, and in the visible range, preferably in the 405-680 nm portion.

In other embodiments, the light sources are replaced by electromagnetic radiation emitters in the farthest infrared range, submillimetric waves or THz radiations of the electromagnetic spectrum, in particular in the 3000-30 μm range, preferably in the 800-50 μm range, and most preferably in the 300-100 μm range. As a matter of fact, it has been experimentally found in international investigations that radiations having such characteristics can propagate in a guided manner inside structures made from a polymeric dielectric material (e.g. S. Atakaramians, S. Afshar V., T. Monro, and D. Abbott, “Terahertz dielectric waveguides,” Adv. Opt. Photon.5, 169-215 (2013)).

The apparatus for biophotonic tissue treatments according to the present invention can be applied to all of those pathologies wherein the use of a light radiation, either visible or in the near infrared region, might be beneficial. It might be useful in treating not only paradontitis and peri-implantitis, but also in treating inflammatory statuses, abscesses, aphtae, post-operative pains, pains from temporomandibular disfunctions, etc. Also, these applications foster healing from injuries, including the operative ones, and stimulate the osteo-integration processes and increase the success rate of implant therapy.

Even though reference has been mainly made to intra-oral treatments in the present description, and in particular to those relevant to the dental and gengival system, the applicator of the apparatus according to the invention might be modified in its shape to make it usable for biophotonic tissue treatments of other body districts. For instance, it might be modeled as a joint immobilizer (including ankle-bands, knee-bands, and the like), collar for cervical traumas, bone fracture brace, bandages for injuries, cuts and/or burns, fingerstall for treating onychomycosis, endovaginal devices for treating lesions from papilloma virus. For all of these applications, the applicator will be implemented, for instance, by way of a 3D molding, so as to present a closed cavity and confine the light guides therein.

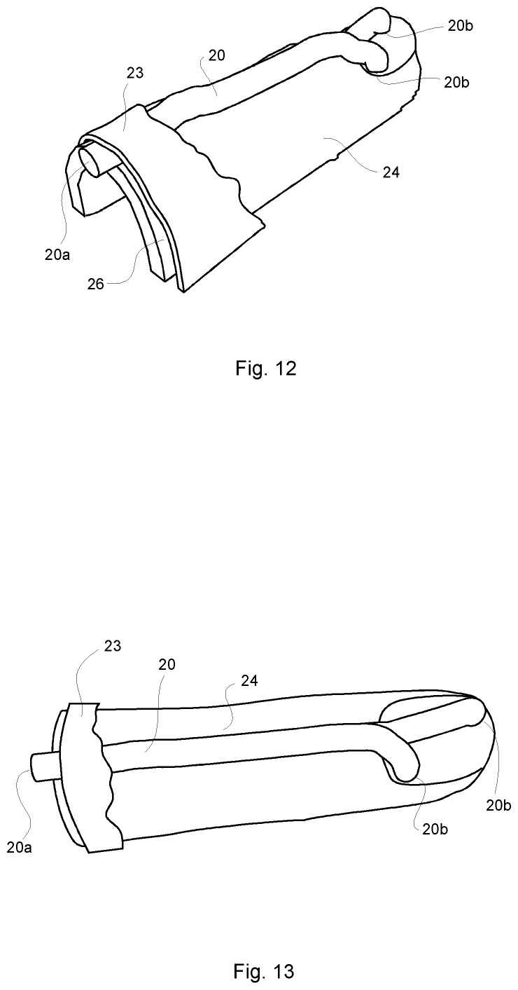

FIG. 12 shows a possible implementation of a fingerstall for treating onychomycosis. The fingerstall is formed of a first shell 23 and a second shell 24 closed on one another, so as to delimit a cavity or inner chamber 26 and is shaped so as to be possibly applied to a finger to be treated. The second shell 24 is in contact with the finger and the part to be treated, whereas the first shell 23 is only partially shown in the figure for the sake of illustrative convenience. Light guides 20 extend inside the chamber 26 on the first shell 23 and terminate in the second shell 24 at the area to be treated. The proximal end 20a of each light guide 20 connects the optical fibre terminal which connect the applicator to the control unit, as described above. FIG. 13 shows a different view of the above-described fingerstall. The implementation of the fingerstall being customizable and wearable makes it possible to apply a treatment wherever necessary.

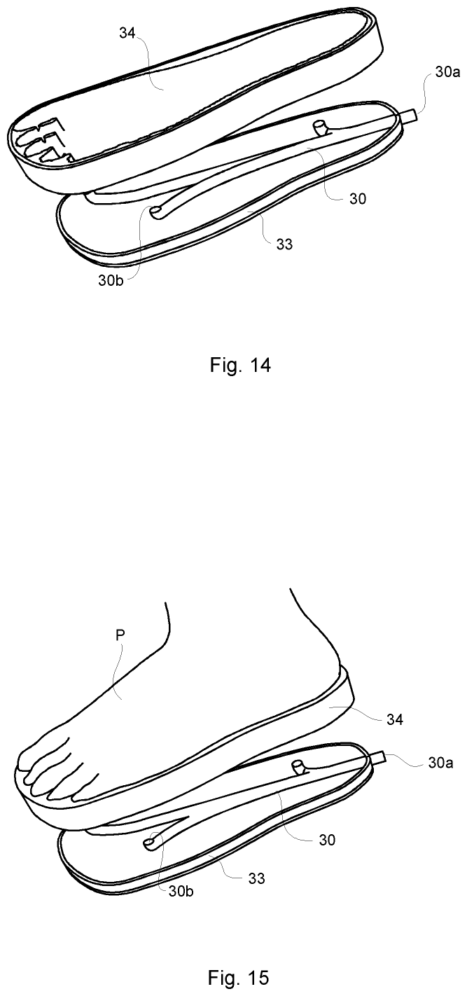

FIG. 14 shows a possible implementation of a shoe sole for a foot, which is incorporated, if necessary, or can be incorporated in a shoe (not shown), for treating foot ulcers and sores and the so-called “diabetic foot” pathology. The shoe sole is formed of a first shell 33 and a second shell 34, closed on one another so as to delimit a cavity or inner chamber 36 and is shaped in such a way as to be possibly applied to the foot to be treated. The second shell 34 is in contact with a foot and the part to be treated, whereas the first shell 33 is shown separated from the second shell 34 for a better illustration of the embodiment. Light guides 30 extend inside the chamber 36 in the first shell 33 and terminate in the second shell 34 at the area to be treated. The proximal end 30a of each light guide 30 connects the optical fibre terminal which connects the applicator to the control unit, as described above.

In FIG. 15, the applicator in the above described form of a shoe sole for a foot is shown applied to a foot P in order to demonstrate its wearability. In this embodiment, the application to the sole of a foot is shown, but the same technique can be adopted to implement an applicator for treating the instep of a foot or a complete foot. The complete or partial implementation of a shoe being customizable and wearable makes it possible to apply a treatment wherever necessary.

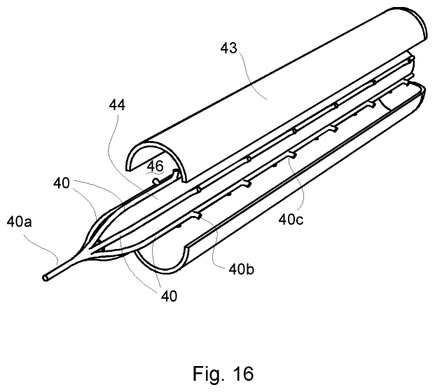

FIG. 16 is an exploded view of a possible embodiment of an endovaginal applicator. The endovaginal applicator is formed of a first shell 43 and a second shell 44 closed on one another to delimit a cavity or inner chamber 46 and is shaped in such a way as to be possibly inserted into a vagina. For this purpose, the second shell 44 has a substantially cylindrical shape, as well as the first shell 43 which, in use, is intended for getting in contact with the endovaginal wall and a part to be treated. In FIG. 16, the first shell 43 is shown subdivided into two parts and separate from the remaining structure for the sake of illustrative clarity. The second shell 44 constitutes a supporting structure for the light guides 40 arranged radially. Arms 40c of the light guides 40 terminate, with their distal ends 40b, in the first shell 43, in contact with the tissues to be treated, and close to the areas to be treated. A connector is mounted on the proximal ends 40a of the light guides 40 for connecting the optical fibre, which allows to connect to the control unit 12, as described above. The applicator being customizable and wearable makes it possible to make a treatment wherever necessary.

Variants and/or modifications might be introduced to the apparatus for biophotonic tissue treatments according to the present invention, without departing from the scope of protection of the invention itself, as set forth in the following claims.

1. An apparatus for biophotonic tissue treatments, characterized in that it comprises a wearable light emitter device, suitable to be configured to a body portion to be submitted to the treatment, said light emitter device comprising a closed chamber, light guides being formed at the inside of said chamber and having distal ends surfacing from said device at prefixed treatment points, and proximal ends merging to an inlet connector where the connection is made between said light guide proximal ends and at least an optical fibre, which directs in said light guides the light generated by at least a light radiation source arranged in a control unit for controlling the emission of said light radiation, said control unit being operated by an operation unit for managing said treatment and supervise the implementation thereof according to set up parameters, said light guides being formed during the same production process and with the same material as said light emitter device.

2. The apparatus according to claim 1, wherein said light emitter device comprises two shells delimiting said closed chamber, said light guides extending within said chamber along one of said shells and ending with their distal ends at said prefixed treatment points defined on the other of said shells.

3. The apparatus according to claim 1, wherein from said light guides light guide arms branch off, whose free ends constitute the distal ends of said light guides ending at said prefixed treatment points.

4. The apparatus according to claim 1, wherein said light emitter device has an arcuate shape and a substantially U-shaped cross section to be fit for being applied to at least a portion of a dental arch.

5. The apparatus according to claim 4, comprising a first shell and a second shell, both being of arcuate shape and substantially U-shaped, the first shell being formed by a base and two side walls extending from opposite sides of the base along the inner part and the outer part of the teeth, said second shell being formed by a base and two side walls extending form opposite sides of the base along the inner part and the outer part of the teeth, said second shell being arranged within said first shell and the relevant bases of the two shells, as well as the relevant side walls and of the two shells, extending in a substantially two-by-two parallel relationship, said chamber being enclosed between said shells.

6. The apparatus according to claim 5, wherein said light guides are formed in said chamber along said side walls of said first shell and branch off towards said prefixed treatment points on the side walls of said second shell.

7. The apparatus according to claim 1, wherein the light source is in the visible part of the spectrum and in the near-infrared, in particular in the range 400 nm-1100 nm, preferably in the ranges 400-450 nm, 530-660 nm and 750 -1100 nm, and particularly preferred in the ranges 400-415 nm, 605-660 nm, and 800-820 nm.

8. The apparatus according to claim 1, wherein the light source of the infrared radiation is such that the flux obtained is in the range 10-200 J/cm², preferably in the range 50-150 J/cm², and more preferably in the range 70-120 J/cm²; the light source of the red radiation is such that the flux obtained is in the range 2-200 J/cm², preferably in the range 5-100 J/cm², and more preferably in the range 7-50 J/cm²; the light source of the violet radiation is such that the flux obtained is in the range 10-200 J/cm², preferably in the range 30-100 J/cm², and more preferably in the range 50-75 J/cm².

9. The apparatus according to claim 1, wherein the light guides are used also to carry towards the control unit optical signals useful to diagnostic purposes, such as mid-wavelength infrared, preferably in the range 10-2 nm, near-infrared, preferably in the range 750-980 nm, and in the visible spectrum, preferably in the range 405-680 nm, to determine temperature, inflammatory condition, contamination, and the like, of the tissue under treatment, means being provided to process said signals to control the radiation emission during the treatment, to vary radiation parameters by automatically correcting them on the basis of the information received.

10. The apparatus according to claim 1, wherein said light emitter device is in the form of a fingerstall for biophotonic treatment of onychomycosis, said fingerstall being formed by a first shell and a second shell closed on another to delimit an inner chamber, said second shell being configured to contact a finger and the finger part to be treated, light guides being formed inside said chamber on said first shell and ending on said second shell at the part to be treated.

11. The apparatus according to claim 1, wherein said light emitter device is in the form of a shoe sole formed by a first shell and a second shell closed on one another to delimit an inner chamber, the second shell being configured to contact a tooth and the part to be treated, light guides being formed inside said chamber on said first shell and ending on said second shell at the part to be treated.

12. The apparatus according to claim 1, wherein said light emitter device is formed by a first shell and a second shell closed on one to another to delimit an inner chamber and is configured for being introduced in the vagina, the second shell being of a substantial cylindrical shape as well as the first shell, which, in use, is intended to contact the endovaginal wall and the part to be treated, said second shell being a support for radially arranged light guides, arms of said light guides ending with distal ends thereof on said first shell, in contact to the tissues to be treated, and near the areas to be treated.

13. The apparatus according to any one of the claim 1, wherein the light source is in the part of the farthest infrared, sub-millimetric wave or THz radiation of the electromagnetic spectrum, in particular in the range 3000-30 μm, preferably in the range 800-50 μ, and in a particular preferred way in the range 300 100 μm.

14. The apparatus according to claim 2, wherein from said light guides light guide arms branch off, whose free ends constitute the distal ends of said light guides ending at said prefixed treatment points.

15. The apparatus according to claim 2, wherein the light source is in the visible part of the spectrum and in the near-infrared, in particular in the range 400 nm-1100 nm, preferably in the ranges 400-450 nm, 530-660 nm and 750-1100 nm, and particularly preferred in the ranges 400-415 nm, 605-660 nm, and 800-820 nm.

16. The apparatus according to claim 2, wherein the light source of the infrared radiation is such that the flux obtained is in the range 10-200 J/cm², preferably in the range 50-150 J/cm², and more preferably in the range 70-120 J/cm²; the light source of the red radiation is such that the flux obtained is in the range 2-200 J/cm², preferably in the range 5-100 J/cm², and more preferably in the range 7-50 J/cm²; the light source of the violet radiation is such that the flux obtained is in the range 10-200 J/cm², preferably in the range 30-100 J/cm², and more preferably in the range 50-75 J/cm².

17. The apparatus according to claim 2, wherein the light guides are used also to carry towards the control unit optical signals useful to diagnostic purposes, such as mid-wavelength infrared, preferably in the range 10-2 nm, near-infrared, preferably in the range 750-980 nm, and in the visible spectrum, preferably in the range 405-680 nm, to determine temperature, inflammatory condition, contamination, and the like, of the tissue under treatment, means being provided to process said signals to control the radiation emission during the treatment, to vary radiation parameters by automatically correcting them on the basis of the information received.

18. The apparatus according to claim 3, wherein the light source is in the visible part of the spectrum and in the near-infrared, in particular in the range 400 nm-1100 nm, preferably in the ranges 400-450 nm, 530-660 nm and 750-1100 nm, and particularly preferred in the ranges 400-415 nm, 605-660 nm, and 800 -820 nm.

19. The apparatus according to claim 3, wherein the light source of the infrared radiation is such that the flux obtained is in the range 10-200 J/cm², preferably in the range 50-150 J/cm², and more preferably in the range 70-120 J/cm²; the light source of the red radiation is such that the flux obtained is in the range 2-200 J/cm², preferably in the range 5-100 J/cm², and more preferably in the range 7-50 J/cm²; the light source of the violet radiation is such that the flux obtained is in the range 10-200 J/cm², preferably in the range 30-100 J/cm², and more preferably in the range 50-75 J/cm².

20. The apparatus according to claim 3, wherein the light guides are used also to carry towards the control unit optical signals useful to diagnostic purposes, such as mid-wavelength infrared, preferably in the range 10-2 nm, near-infrared, preferably in the range 750-980 nm, and in the visible spectrum, preferably in the range 405-680 nm, to determine temperature, inflammatory condition, contamination, and the like, of the tissue under treatment, means being provided to process said signals to control the radiation emission during the treatment, to vary radiation parameters by automatically correcting them on the basis of the information received.
